(19) United States
(12) Patent Application Publication
(10) Pub. No.: US 2021/0138099 A1
(43) Pub. Date: May 13, 2021

(54) METHOD OF CONCEALING OUTDOOR EQUIPMENT

(71) Applicant: ScentLok Technologies, Inc., Muskegon, MI (US)

(72) Inventors: Nicholas C. Andrews, Muskegon, MI (US); Greg Paquin, Muskegon, MI (US)

(21) Appl. No.: 17/096,009

(22) Filed: Nov. 12, 2020

Related U.S. Application Data
(60) Provisional application No. 62/934,812, filed on Nov. 13, 2019.

(51) Int. Cl. A61L 2/20; A01M 31/00

(52) U.S. Cl. CPC A61L 2/202; A61L 2202/11; A01M 31/00

(57) ABSTRACT

A method of neutralizing an odor associated contaminant from outdoor equipment includes providing an ozone spray generating unit that generates and sprays a mixture of ozone and water, transporting the ozone spray generating unit into a field environment, generating, by the ozone spray generating unit, the mixture within the ozone spray generating unit while the ozone spray generating unit is present in the field environment, and providing, by the ozone spray generating unit, a spray of the mixture onto the outdoor equipment such that the mixture neutralizes the contaminant, reducing an odor associated with the contaminant. The field environment is an outdoor environment containing an outdoorsman and an animal sought by the outdoorsman. At least one of (a) the odor associated with the contaminant is foreign to the field environment or (b) a relative amount of the odor associated with the contaminant is foreign to the field environment.

### CROSS-REFERENCE TO RELATED PATENT APPLICATION

This application claims the benefit of U.S. Provisional Patent Application No. 62/934,812, filed Nov. 13, 2019, which is incorporated herein by reference in its entirety.

### BACKGROUND

The present disclosure relates generally to systems and methods for eliminating odors. More specifically, the present disclosure relates to eliminating odors in outdoor applications involving wild game.

Humans produce, expel, facilitate the growth of, or otherwise provide substances that emit a spectrum of odors in the form of odor particles. The rate of production of such substances and, by extension the magnitude of the odors that they emit, increases in hot conditions or when humans participate in exercise. However, even at low magnitudes, the odors produced by such substances can be detected by the acute senses of smell of various animals. Outdoorsmen (e.g., hunters, fishermen, field biologists, nature photographers, etc.) often rely on remaining undetected by target species (e.g., deer, bears, etc.) during hunting, fishing or photographing in a natural environment, or in other applications.

Carbon is widely recognized as an odor-controlling material and is used in various industries to eliminate odors, including the animal photography field, the military clothing field, and the hunting, fishing and camouflage fields for changing a human's odor profile. However, the use of carbon alone can be improved upon by attacking the fullest spectrum of human odor detectable as found on a wide range of equipment used by the outdoorsman, such as hunting equipment (e.g., bows, firearms, blinds, tree stands, etc.) or photography equipment (e.g., lenses, camera bodies, etc.).

### SUMMARY

At least one embodiment relates to a method of neutralizing an odor associated contaminant from outdoor equipment. The method includes providing an ozone spray generating unit configured to generate and spray a mixture of ozone and water, transporting the ozone spray generating unit into a field environment, generating, by the ozone spray generating unit, the mixture within the ozone spray generating unit while the ozone spray generating unit is present in the field environment, and providing, by the ozone spray generating unit, a spray of the mixture onto the outdoor equipment in the field environment such that the mixture neutralizes the contaminant from the outdoor equipment, reducing an odor associated with the contaminant. The field environment is an outdoor environment containing an outdoorsman and an animal sought by the outdoorsman. At least one of (a) the odor associated with the contaminant is foreign to the field environment or (b) a relative amount of the odor associated with the contaminant is foreign to the field environment.

Another embodiment relates to a method of concealing a piece of outdoor equipment in a field environment. The method includes providing a contaminant neutralizing unit within the field environment. The contaminant neutralizing unit includes a tank containing water, an ozone generator coupled to the tank, a nozzle coupled to the tank, and a pump fluidly coupled to the tank and the nozzle. The method further includes supplying, by the ozone generator, ozone into the tank while the contaminant neutralizing unit is present within the field environment, holding, by a hand of an outdoorsman, the contaminant neutralizing unit with the nozzle oriented toward the piece of outdoor equipment, and activating the pump to move the water and the ozone from the tank, through the nozzle, and onto the piece of outdoor equipment in the field environment. The field environment is an outdoor environment containing the outdoorsman and an animal sought by the outdoorsman.

Yet another embodiment relates to a method of concealing outdoor equipment in a field environment. The method includes providing a handheld spray unit in the field environment. The handheld spray unit includes a tank containing water. The method further includes applying a voltage across a pair of electrodes in contact with the water to generate ozone that mixes with the water in the tank while the handheld spray unit is present in the field environment, and stopping the generation of the ozone in response to at least one of (a) an indication that a predetermined period of time has elapsed since beginning the generation of the ozone, (b) an indication that a concentration of ozone within the tank has reached a threshold concentration, or (c) an indication that a threshold level of water is no longer present in the ozone spray generating unit. The method further includes holding, by a hand of an outdoorsman, the handheld spray unit such that a nozzle of the handheld spray unit is oriented toward the outdoor equipment, and spraying, from the nozzle, the water and the ozone onto the outdoor equipment in the field environment such that the ozone reduces an odor associated with the outdoor equipment. The field environment is an outdoor environment containing an animal sought by the outdoorsman.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE FIGURES

FIG. 1 is a front perspective view of an ozone spray generation unit, according to an exemplary embodiment.

FIG. 2 is a rear view of the ozone spray generation unit of FIG. 1.

FIG. 3 is a schematic view of the ozone spray generation unit of FIG. 1.

FIG. 4 is a front perspective view of the ozone spray generation unit of FIG. 1 with certain components shown as transparent.

FIG. 5 is a front perspective view of the ozone spray generation unit of FIG. 1 in use by an outdoorsman in a field environment.

FIG. 6 is a block diagram of a method of using an ozone spray generation unit, according to an exemplary embodiment.

FIG. 7 is a front perspective view of a tank and an ozone generator of the ozone spray generation unit of FIG. 1.

FIG. 8 is a bottom perspective view of a spray head of the ozone spray generation unit of FIG. 1.

FIG. 9 is a block diagram of a field environment, according to an exemplary embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

As utilized herein, the term “mixture” means a combination or blend of two or more things (e.g., materials, fluids, gasses, a fluid and a gas, etc.). The mixture may be a homogeneous mixture. By way of example, in a mixture of water and ozone, the ozone may be dissolved into the water such that the ozone and the water form a solution. Alternatively, the mixture may be a heterogeneous mixture. By way of example, in a mixture of water and ozone, the ozone may exist as bubbles in liquid water.

As utilized herein, the term “animal” can include, but is not limited to, mammals (e.g., deer, moose, elk, antelope, raccoons, squirrels, etc.), birds (e.g., geese, pheasants, ducks, etc.), fish, amphibians, and reptiles.

Referring generally to the figures, an ozone spray generating unit is used to neutralize an odor associated contaminant. Odor associated contaminants may include odor-emitting substances, odor particles, bacteria, dust mites, mold, and/or other substances or organisms that are associated with (e.g., produce, expel, etc.) odors. The ozone spray generating unit includes a tank or reservoir configured to receive water intended to be acted upon by an ozone generator. The ozone generator is configured to generate ozone, which is then mixed with the water for application to gear, clothing, structure, or equipment associated with an odor associated contaminant. The water is drawn out of the tank, and the mixture of ozone and water is forced out through a nozzle to generate a spray. The ozone may be mixed with the water within the tank, or the ozone may be mixed with the water while the water flows toward the nozzle. The ozone within the water is configured to neutralize one or more odor associated contaminants, reducing the presence of undesirable odors (e.g., undesirable scents) and/or disinfecting one or more items.

In some embodiments, the spray of the mixture of ozone and water is used to neutralize contaminants on outdoor equipment used by an outdoorsman. Preferably, the unit is transported to a field environment and filled with water, but water can be added once the unit is in the field. The ozone generator is activated to ozonate the water in the bottle or reservoir. The outdoorsman then produces a spray of the water and ozone mixture using the unit and directs the spray onto the outdoor equipment, gear, clothing or other item containing the contaminant. The mixture neutralizes odor associated contaminants on the outdoor equipment that could otherwise produce odors that would alert animals to the presence of the outdoorsman. The unit is sized to facilitate transportation into the field environment. Ozone will not remain in solution in water indefinitely and therefore, it can be beneficial to wait to generate the ozone until arriving in the field. By utilizing the unit to neutralize odor associated contaminants after the outdoor equipment is transported into the field environment, this ensures that a maximum amount of contaminants are neutralized. The unit is also able to neutralize odor associated contaminants associated with virtually any equipment used in the field and does not require the equipment to be purpose-built to reduce odors.

Referring to FIGS. 1-5,7, and 8, a self-contained ozone mist generating unit, a self-contained contaminant neutralizing unit, handheld spray unit, or ozone spray generating unit, shown as unit 10, is shown according to an exemplary embodiment. The unit 10 is configured to generate a spray of a mixture of ozone and a liquid, preferably water, to neutralize (e.g., destroy, reduce the prevalence of, reduce the detectability of, kill, etc.) one or more contaminants (e.g., odor-emitting substances, odor particles, bacteria, dust mites, mold, and/or other undesirable substances or organisms). The unit 10 includes a spray portion or head portion, shown as spray head 12, a body portion or container (e.g., a tank, a bottle, a vessel, a reservoir, etc.), shown as tank 14 preferably positioned immediately below the spray head 12, and a neutralizer generation portion or ozone generation portion, shown as ozone generator 16 preferably adjacent to the tank 14. As shown, the tank 14 extends between and is coupled to the spray head 12 and the ozone generator 16. In operation, the tank 14 receives and contains a volume of water W, and the ozone generator 16 uses the water W as the source for the ozone created, but as described below, also uses the water W as the medium for carrying and distributing the ozone onto the article to be deodorized. Preferably, the ozone generator 16 creates ozone in a molecular, rather than gaseous form so that the ozone is dissolved in solution in the mixture. However, the ozone generator 16 can instead be adopted to generate ozone in gaseous form so that ozone bubbles are provided in the water W within the tank 14. The spray head 12 is activated by a user to generate a spray of the mixture, which a user directs toward one or more contaminants to be neutralized.

Referring to FIGS. 1-4 and 8, the spray head 12 includes a housing, shown as body 20. The body 20 is coupled to the tank 14. In some embodiments, the body 20 is selectively coupled to the tank 14 such that the spray head 12 can be removed from the tank 14 (e.g., to permit filling of the tank 14 with the water W). By way of example, the body 20 and the tank 14 may have corresponding threaded portions that engage one another to selectively couple the body 20 and the tank 14 together. As shown in FIGS. 7 and 8, the tank 14 defines a male thread, and the body 20 of the spray head 12 defines a female thread configured to engage with the male thread of the tank 14. However, it is understood that any number of conventional means for securing the spray head 12 to the tank 14 can be used, including, but not limited to a snap fit or friction fit.

The spray head 12 includes a fluid impeller, flow controller, or fluid flow device, shown as pump 22. The pump 22 is fluidly coupled to a stream shaper, shown as nozzle 24, that defines an outlet 26. The pump 22 is configured to be activated by a user interface (e.g., a button, a switch, a trigger, a lever, etc.), shown as trigger 28. Accordingly, the trigger 28 may be coupled to the pump 22. When a user interacts with (e.g., engages, pulls, pushes, depresses, etc.) the trigger 28, the pump 22 is activated. When activated, the pump 22 is configured to draw the mixture of ozone and water W from the tank 14 and force the mixture out of the spray head 12 through the outlet 26 of the nozzle 24. The pump 22 may be electrically actuated. By way of example, the trigger 28 may be coupled to a switch that selectively supplies electrical energy to drive a motor of the pump 22. Alternatively, the pump 22 may be mechanically actuated. By way of example, the trigger 28 may be coupled to a piston of the pump 22 such that movement of the trigger 28 causes a corresponding movement of the piston, driving the mixture through the pump 22. In some embodiments, the spray head 12 includes one or more conduits (e.g., tubes, hoses, pipes, etc.), shown as hose 30, through which the mixture is drawn into the pump 22. The hose 30 may extend to near the bottom of the tank 14 to facilitate drawing material into the pump 22 when the level of the water W within the tank 14 is low.

The nozzle 24 is configured (e.g., shaped, sized, etc.) to generate a fine spray of the mixture. The size (e.g., area covered by the spray at a certain distance from the nozzle 24, an overall width, height, or diameter of the spray at a certain distance from the nozzle 24, etc.), shape (e.g., a cross-sectional shape of the stream), and droplet size of the spray may vary between different embodiments. In some embodiments, the nozzle 24 is configured such that the stream is a fine mist having many small droplets. Such a mist may exit the nozzle 24 in a substantially conical pattern. In some embodiments, the size of the stream is adjustable (e.g., by rotating the nozzle 24 to change a size of the outlet 26). In some embodiments, the nozzle 24 is configured such that the spray is a concentrated stream. Such a concentrated stream may have a substantially constant cross-sectional size as the stream extends away from the nozzle 24. In some embodiments, the pump 22 and the nozzle 24 are configured to provide a continuous stream of the mixture (e.g., when the user continuously presses the trigger 28, when a user presses the trigger 28 at a high frequency, etc.).

Referring again to FIGS. 1-4 and 7, the tank 14 defines an inlet and/or outlet, shown as filling aperture 40, through which the pump 22 and the hose 30 extend into the tank 14 when the spray head 12 is coupled to the tank 14. The body 20 of the spray head 12 may sealingly engage the body 50 to form a sealed volume between the tank 14 and the spray head 12. This seal ideally prevents the water W and/or the ozone from exiting the unit 10, except through the nozzle 24. When the spray head 12 is removed, the filling aperture 40 may be open to permit adding additional water W to the tank 14.

Referring again to FIGS. 1-4, the ozone generator 16 includes a housing, frame, chassis, or body, shown as body 50. The body 50 may be coupled to and support the other components of the ozone generator 16. The body 50 is coupled to a bottom end of the tank 14. In some embodiments, the body 50 is selectively coupled to the tank 14. By way of example, the body 50 and the tank 14 may be in threaded engagement with one another.

The ozone generator 16 includes a controller 52, which includes a processor 54 and a memory device, shown as memory 56. The controller 52 is configured to control the operation of the ozone generator 16. The controller 52 is operatively coupled to a user interface device (e.g., a button, a switch, a touchscreen, a knob, a dial, a light, a speaker, a vibrating motor, etc.), shown as user interface 60. The user interface 60 may be configured to provide information (e.g., commands) to the controller 52. As shown in FIGS. 4 and 7, the user interface 60 includes a user interface device, shown as button 59, configured to command the controller 52 to begin generation of ozone when activated or pressed. In some embodiments, the controller 52 is configured to automatically stop the generation of ozone after a predetermined, threshold period of time (e.g., 5 seconds, 10 seconds, 60 seconds, etc.) has elapsed since the beginning of the generation of ozone. As described further below, in other embodiments, the controller is configured to automatically stop the generation of ozone once a predetermined threshold of ozone concentration has been reached in the solution of water. In some embodiments, the controller 52 is configured to automatically stop the generation of ozone in response to an indication that a threshold level of water is no longer present in the tank 40 (e.g., the tank 40 includes no water, the water within the tank 40 has fallen below a predetermined fill level, etc.). Additionally or alternatively, the user interface 60 may be operated by the controller 52 to provide information to a user. As shown in FIGS. 4 and 7, the user interface 60 includes user interface devices, shown as lights 61, that are illuminated when ozone is being generated. Specifically, a first light 61 is positioned within the tank 14, and a second light 61 surrounds the button 59, providing visual information to the user from multiple viewing angles. Placing a light 61 within the tank 14 may illuminate the water W, further increasing the visibility of any information provided by the lights 61. In other embodiments, the user interface 60 is omitted.

The ozone generator 16 further includes a sensor 62 operatively coupled to the controller 52 and is configured to provide information to the controller 52. In some embodiments, the sensor 62 detects a characteristic, property, or state of the mixture within the tank 14. By way of example, the sensor 62 may be configured to provide information relating to a relative concentration of ozone within the mixture. By way of example, the sensor 62 may be configured to indicate an amount of water within the tank 14. The controller 52 may then control operation of the ozone generator 16 based at least in part on this information. By way of example, the controller 52 may be configured to stop generation of ozone when the concentration of ozone in the mixture reaches a threshold level. By way of another example, the controller 52 may be configured to prevent generation of ozone when less than a threshold amount of water W is provided in the tank 15 (e.g., when there is no water W left in the tank 14).

The ozone generator 16 further includes a power source 70 configured to supply electrical energy (e.g., alternating current, direct current) to power the controller 52 and other components of the ozone generator 16. The power source 70 may be or include an energy storage device (e.g., batteries, capacitors, supercapacitors, etc.), an energy generation device (e.g., a solar panel, a piezoelectric element, etc.), or a connection to another source of electrical energy (e.g., a USB port, etc.). In some embodiments, the power source 70 includes an energy storage device that is configured to be recharged with an outside source of electrical energy (e.g., an electrical grid, a generator, etc.). In embodiments where the pump 22 is electrically actuated, the power source 70 may be coupled to the pump 22 and/or the trigger 28 and configured to selectively provide electrical energy to operate the pump 22.

In the embodiment shown in FIGS. 3 and 4, the ozone generator 16 includes a pair of conductors, shown as electrodes 80, operatively coupled to the controller 52. The electrodes 80 are positioned to be in direct contact with the water W. In some embodiments, such as the embodiment shown in FIG. 3, the electrodes 80 extend into the tank 14. In other embodiments, such as the embodiment shown in FIG. 4, the electrodes 80 are located entirely within the body 50, and the electrodes 80 are fluidly coupled to the sealed volume of the tank 14. Specifically, as shown in FIGS. 4 and 7, the electrodes 80 are separated from the tank 14 by a sheet of perforated material, shown as grate 82, that permits water W from the tank 14 to flow into the body 50.

In some embodiments, the controller 52 is configured to apply a voltage (e.g., supplied by the power source 70) across the electrodes 80 such that a current passes through one electrode 80, the water W, and the other electrode 80. When the controller 52 applies the voltage across the electrodes 80, one of the electrodes 80 acts as an anode (i.e., is positively charged), and the other electrode 80 acts as a cathode (i.e., is negatively charged). In some embodiments, the voltage applied by the controller 52 has a constant polarity, such that the roles of the electrodes 80 as the anode and cathode, respectively, are constant. In other embodiments, the controller 52 routinely varies the polarity of the applied voltage, changing the roles of the electrodes 80. Certain types of build-up or wear may be more severe or solely apply to the anode or the cathode. By changing the polarity, build-up or wear may be reduced.

When the controller 52 applies the voltage across the electrodes 80 through the water W, electrolysis occurs, consuming water molecules (H₂O) and generating (e.g., precipitating) ozone molecules (O₃). Under these circumstances, different reactions occur at the anode and the cathode. Specifically, a primary anode reaction, shown in Equation 1, occurs at the anode. In the primary anode reaction, water (H₂O) is consumed, and ozone (O₃), monatomic hydrogen (H), and free electrons (e⁻) are produced.

3H₂O→O₃+6H+6e⁻ (1)

A secondary anode reaction, shown in Equation 2, also occurs at the anode. In the secondary anode reaction, water (H₂O) is consumed, and diatomic oxygen (O₂), monatomic hydrogen (H), and free electrons (e⁻) are produced.

2H₂O→O₂+4H+4e⁻ (2)

One or more reactions that occur at the cathode may consume the products of the anode reactions. Specifically, these reactions may consume certain products of the anode reactions without consuming the generated ozone. The cathode may initiate a hydrogen evolution cathode reaction, shown in Equation 3. In the hydrogen evolution cathode reaction, monatomic hydrogen (H) and free electrons (e⁻) are consumed, and diatomic hydrogen (H2) is produced.

2H+2e⁻→H₂ (³)

Additionally or alternatively, the cathode may initiate an oxygen reduction cathode reaction, shown in Equation 4. In the oxygen reduction cathode reaction, diatomic oxygen (O₂), monatomic hydrogen (H), and free electrons (e⁻) are consumed, and water (H₂O) is produced. The produced water may subsequently be consumed by the primary anode reaction to generate additional ozone or by the secondary anode reaction.

2O₂+4H+4e^{-→}2H₂O (4)

In one embodiment, the ozone molecules generated by the primary anode reaction are dissolved within the remaining water W to form the mixture. The diatomic hydrogen generated by the hydrogen evolution cathode reaction typically forms gas bubbles within the water W. These bubbles of diatomic hydrogen may rise through the water W to gather at the top of the tank 14. In other embodiments, the ozone molecules may form bubbles within the water W. These bubbles of ozone may be mixed within the water W to form the mixture for application to the contaminants.

In some embodiments, one electrode 80 is made from (e.g., made entirely from, made primarily from, includes) a first material, and the other electrode 80 is made from a different, second material. In embodiments where the electrodes 80 are made from different materials, the controller 52 may apply a voltage having a constant polarity across the electrodes 80 such that the electrode 80 of the first material consistently acts as the anode, and the electrode 80 of the second material consistently acts as the cathode. In one embodiment, one electrode 80 is made from platinum, and the other electrode 80 is made from titanium. In other embodiments, the electrodes 80 are made from the same material.

In an alternative embodiment, the electrodes 80 are positioned along the flow path of water W from the tank 14 to the nozzle 24. By way of example, the electrodes 80 may be positioned within the hose 30, within the pump 22, or between the pump 22 and the nozzle 24. In such embodiments, the electrodes 80 may produce ozone within the water W as the water W passes out of the tank 14 to the nozzle 24. Accordingly, the ozone generator 16 may mix ozone with a small portion of the water W as opposed to mixing ozone with all of the water W contained in the tank 14 at once.

In other embodiments, the ozone generator 16 performs a different type of ozone generation, specifically, the ozone generator 16 generates ozone from atmospheric air or diatomic oxygen (O₂). By way of example, the ozone generator 16 may generate ozone by exposing air to ultraviolet light. By way of another example, the ozone generator 16 may generate ozone through corona discharge.

Referring to FIGS. 1 and 5, the unit 10 is shaped to facilitate handheld use by an operator. The unit 10 extends along a substantially central vertical axis 90. Specifically, the spray head 12, the tank 14, and the ozone generator 16 are approximately centered about the axis 90. In use, an operator may wrap their hand around a bottom portion of the spray head 12 and a top portion of the tank 14, with one or more fingers (e.g., an index finger, an index finger and a middle finger, etc.) engaging (e.g., resting on, pressing against, etc.) the trigger 28. To facilitate this grip position, a cross-section (e.g., as viewed in relation to the axis 90) of the tank 14 decreases near the top of the tank, and a cross-section of the spray head 12 decreases near the bottom of the spray head 12. This may facilitate guiding the operator's hand to a position in which the user may easily actuate the trigger 28. To initiate a spray of the mixture from the unit 10, the operator may squeeze or press the trigger 28 toward the axis 90.

The unit 10 is also configured to facilitate portability. The unit 10 is sized to easily fit into a bag or to be carried by a user. By way of example, a hunter may transport the unit 10 to a field environment (commonly referred to as “in the field”) within a container including the rest their outdoor (e.g., hunting or fishing) equipment. By way of another example, a hunter or fisherman may transport the unit 10 into a location where the user intends to hunt or fish (e.g., as shown in FIG. 5, into a tree stand, into a duck blind, or along a shoreline, etc.). This may facilitate reapplying the spray to their equipment during the activity if odors become detectable or otherwise rise to an undesirable level. The unit 10 is preferably self-contained and does not require any outside resources (e.g., electrical energy, water, etc.) during an operational period, further facilitating portability.

Referring to FIG. 6, a method of using the unit 10 is illustrated, according to an exemplary embodiment. Specifically, FIG. 6 illustrates a method 200 of using the unit 10 to eliminate contaminants from outdoor equipment. The method 200 may be performed by an outdoorsman (e.g., a hunter, a wildlife photographer, a birdwatcher, a hiker, a fisherman, etc.). As used herein, the term “outdoorsman” may include a person of any gender. In some embodiments, the outdoorsman seeks one or more animals in a field environment. The field environment may be any outdoor environment containing animals (e.g., mammals, birds, fish, reptiles, or amphibians). By way of example, a field environment may include forests, plains, fields, meadows, tundra, savannah, lakes, rivers, oceans, or other outdoor environments.

Certain odor-causing contaminants may emit odors that are foreign or unusual to the field environment or may be present at an amount of an odor that is foreign or unusual to the field environment. By way of example, a contaminant may emit an odor that is not normally present in the field environment, or an odor that is only present in the field environment in small quantities. Such odors may alert the animals to the presence of the outdoorsman if the odors are detected by the animals. These odor-causing contaminants may be produced and/or carried by the outdoorsman (e.g., sweat, oils, breath, etc.). The outdoorsman may then transfer (e.g., by touch, by breathing, etc.) the contaminants onto outdoor equipment that is used by the outdoorsman. Alternatively, the outdoor equipment may receive the contaminants from another source (e.g., coming into contact with another object prior to being transported into the field environment, coming into contact with a scented cleaning product, etc.). Further alternatively, the outdoor equipment may inherently have certain contaminants (e.g., certain materials may emit odors, etc.).

The outdoorsman may employ the method 200 to neutralize one or more odor-causing contaminants, reducing or eliminating an odor associated with the contaminant. Reducing the odor may reduce a likelihood that the outdoorsman is detected by the animals. The outdoorsman may then be able to more easily access the sought animals without detection (e.g., is able to get closer without detection than would otherwise be possible, is able to remain near the animals without detection for a longer period than would otherwise be possible, etc.).

In some embodiments, the outdoorsman is a hunter seeking (e.g., tracking, hunting, etc.) one or more game animals (e.g., deer, moose, elk, antelope, geese, pheasants, ducks, etc.). In such embodiments, the outdoor equipment may be hunting equipment. The hunting equipment may include clothing worn by the hunter (e.g., shoes (e.g., boots), shirts, pants, socks, hats, masks, gloves, etc.). The hunting equipment may include equipment used to injure or impede the movement of the sought game animals. By way of example, the hunting equipment may include bows, arrows, firearms, and/or ammunition. The hunting equipment may also include a structure configured to support or conceal the hunter. By way of example, the hunting equipment may include tree stands, duck blinds, or seats (e.g., chairs, stools, etc.).

In other embodiments, the outdoorsman practices another type of outdoor activity. By way of example, the outdoorsman may be a birdwatcher, a wildlife photographer, a hiker, a scientist, or another type of outdoor enthusiast. The outdoorsman may utilize any outdoor equipment used by the hunter as well as other types of clothing, photography equipment (e.g., lenses, camera bodies, etc.), or other types of equipment.

In step 202 of the method 200, the unit 10 is transported into a field environment. The unit 10 may be transported by an outdoorsman. By way of example, the unit 10 may be transported by hand, in a storage container (e.g., a backpack, a duffel, etc.), or in a vehicle.

In step 204 of the method, water is provided into the tank 14 of the unit 10. In some embodiments, the spray head 12 is removed from the tank 14, water is provided through the filling aperture 40, and the spray head 12 is reattached to the tank 14. The water may contain a negligible amount of ozone when added to the tank 14. The water may be purified or unpurified water. When the ozone is generated and mixed with the water, contaminants within the water may be neutralized. Step 204 may be performed before or after the unit 10 is transported to the field. In embodiments where step 204 is performed prior to transportation into the field, the water may be provided by any source (e.g., tap water from a well or municipal grid, bottled water, etc.). In embodiments where step 204 is performed in the field, the water may be provided by any available source within the field environment (e.g., bottled water transported into the field, a stream, rainwater, etc.).

In step 206, the unit 10 generates the mixture of water and ozone within the ozone spray generating unit 10. In some embodiments, the unit 10 begins generating the mixture in response to a user input (e.g., a button press, etc.) through the user interface 60. In one embodiment, the controller 52 applies a voltage across the electrodes 80 to generate ozone within the water pursuant to the processes and reactions described above. The water may remain within the tank 14 while the ozone is generated. In some embodiments, the controller 52 continues to generate ozone within the water until receiving an indication (e.g., from a clock or timer within the controller 52) that a threshold period of time has elapsed since beginning the generation of ozone). In other embodiments, the controller 52 continues to generate ozone within the water until receiving an indication (e.g., from the sensor 62) that a concentration of ozone in the mixture within the tank 14 has reached a desired threshold concentration. In both such types of embodiments, most or all of the water within the unit 10 is mixed with ozone at one time. Accordingly, the outdoorsman may then distribute the mixture having the desired ozone concentration. In response to completion of the generation of the ozone, the controller 52 may provide a notification (e.g., a light, a sound, etc.) to the outdoorsman (e.g., through the user interface 60).

In step 208, the outdoorsman directs or provides a spray of the mixture of water and ozone onto one or more pieces of outdoor equipment. The outdoorsman may select one or more pieces of outdoor equipment to spray with the mixture. The outdoorsman may hold the unit 10 in their hand and orient the outlet 26 of the nozzle 24 toward the desired outdoor equipment for treatment. FIG. 5 illustrates a user (e.g., a hunter), shown as outdoorsman 100, utilizing the unit 10 in a field environment 102, according to an exemplary embodiment. Specifically, the outdoorsman 100 holds the unit 10 in a first hand, shown as right hand 104. The outdoorsman 100 uses a second hand, shown as left hand 106, to hold a piece of outdoor equipment, shown as bow 108. The outdoorsman 100 orients the unit 10 such that the outlet 26 of the nozzle 24 is oriented toward the bow 108.

The outdoorsman may initiate the spray of the mixture by squeezing or pressing the trigger 28. This causes the pump 22 to draw the mixture from the tank 14 into the spray head 12. The pump 22 then forces the mixture toward the nozzle 24. The outlet 26 of the nozzle 24 then defines a spray of the mixture outward from the spray head 12. The mixture then contacts the outdoor equipment (e.g., the bow 108), such that the ozone engages and neutralizes contaminants within and on the outer surface of the outdoor equipment.

As the unit 10 sprays the mixture, the outdoorsman may rotate or translate the unit 10 and/or the outdoor equipment to spray different areas of the outdoor equipment with the mixture. The outdoorsman will continue this process until the most or all of the surface of the outdoor equipment has been covered with the mixture. The outdoorsman may select another piece of outdoor equipment and repeat step 208 until the contaminants have been sufficiently neutralized. If the outdoorsman determines that additional contaminants may again need to be neutralized (e.g., detects an odor, etc.), the outdoorsman may repeat step 208 on the same piece of outdoor equipment.

If the unit 10 runs out or begins to run out of the mixture, the outdoorsman may add additional water to the tank 14 in step 210. Step 210 may be substantially similar to step 204 as described herein. Once step 210 has been completed, the outdoorsman can repeat steps 206 and 208 as necessary to neutralize additional contaminants.

In an alternative embodiment, the unit 10 may generate ozone after the water leaves the tank 14. In this embodiment, steps 206 and 208 may occur simultaneously. Specifically, the pump 22 may pump water from the tank 14 toward the nozzle 24, and ozone may be generated within the water as the ozone is pumped toward the nozzle 24.

FIG. 9 illustrates a field environment 300, according to an exemplary embodiment. The field environment 300 contains the units 10, outdoorsmen 302 (e.g., hunters, photographers, fishermen, birdwatchers, hikers, etc.), animals 304 (e.g., game animals sought by the outdoorsmen 302), and outdoor equipment 310 (e.g., hunting equipment, equipment worn or otherwise used by the outdoorsmen 302, etc.). The outdoor equipment 310 includes clothing, such as shoes 312, shirts 314, pants 316, socks 318, hats 320, masks 322, and gloves 324. The outdoor equipment 310 includes bows 326, arrows 328, firearms 330, ammunition 332, photography equipment 334 (e.g., cameras, lenses, memory cards, film, tripods, etc.), fishing tackle 336 (e.g., rods, reels, lures, bait, hooks, line, etc.), containers 338 (e.g., bags, crates, boxes, etc.), blinds 340 (e.g., hunting blinds, duck blinds, etc.), tree stands 342, and seats 344 (e.g., chairs, stools, etc.). In other embodiments, one or more of these items are excluded from the field environment 300, and/or the field environment 300 contains one or more additional items.

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the ozone spray generation unit as shown in the various exemplary embodiments is illustrative only. Additionally, any element disclosed in one embodiment may be incorporated or utilized with any other embodiment disclosed herein. For example, the ozone generation procedure of the exemplary embodiment described in at least Paragraph [0040] may be incorporated in the unit 10 of the exemplary embodiment shown in at least FIG. 1. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A method of neutralizing an odor associated contaminant from outdoor equipment, comprising:
providing an ozone spray generating unit configured to generate and spray a mixture of ozone and water;
transporting the ozone spray generating unit into a field environment;
generating, by the ozone spray generating unit, the mixture within the ozone spray generating unit while the ozone spray generating unit is present in the field environment; and
providing, by the ozone spray generating unit, a spray of the mixture onto the outdoor equipment in the field environment such that the mixture neutralizes the contaminant from the outdoor equipment, reducing an odor associated with the contaminant,
wherein the field environment is an outdoor environment containing an outdoorsman and an animal sought by the outdoorsman, and wherein at least one of (a) the odor associated with the contaminant is foreign to the field environment or (b) a relative amount of the odor associated with the contaminant is foreign to the field environment.

2. The method of claim 1, wherein generating, by the ozone spray generating unit, the mixture within the ozone spray generating unit includes:
generating, by an ozone generator of the ozone spray generating unit, the ozone; and
mixing the ozone with the water, wherein the water is provided within a reservoir of the ozone spray generating unit.

3. The method of claim 2, wherein the ozone generator includes a pair of electrodes, and wherein the ozone generator is configured to generate the ozone by applying a voltage across the electrodes while the electrodes are in contact with the water.

4. The method of claim 3, wherein a first electrode of the pair of electrodes is made from titanium, and wherein a second electrode of the pair of electrodes is made from platinum.

5. The method of claim 3, wherein providing, by the ozone spray generating unit, the spray of the mixture includes:
drawing, by a pump of the ozone spray generating unit, the water out of the reservoir and toward a nozzle of the ozone spray generating unit; and
defining, by the nozzle, the spray of the mixture.

6. The method of claim 5, wherein the ozone is mixed with the water in the reservoir such that the pump draws the mixture out of the reservoir.

7. The method of claim 6, further comprising stopping the generation of the mixture in response to at least one of:
an indication that a threshold period of time has elapsed since beginning the generation of the mixture;
an indication that a concentration of ozone within the reservoir has reached a threshold concentration; or
an indication that a threshold level of water is not present in the reservoir.

8. The method of claim 1, wherein the outdoor equipment is hunting equipment, wherein the outdoorsman is a hunter, and wherein the animal is a game animal sought by the hunter.

9. The method of claim 8, wherein the hunting equipment includes clothing configured to be worn by the hunter.

10. The method of claim 8, wherein the hunting equipment includes at least one of a bow, a firearm, an arrow, or ammunition.

11. The method of claim 8, wherein the hunting equipment includes a structure configured to support or conceal the hunter.

12. The method of claim 1, wherein the ozone spray generating unit includes a tank containing the water, further comprising supplying additional water to the tank while in the field environment and after spraying at least a portion of the water from the tank.

13. The method of claim 1, further comprising stopping the generation of the mixture in response to at least one of:
an indication that a threshold period of time has elapsed since beginning the generation of the mixture;
an indication that a concentration of ozone within the mixture has reached a threshold concentration; or
an indication that a threshold level of water is no longer present in the ozone spray generating unit.

14. A method of concealing a piece of outdoor equipment in a field environment, comprising:
providing a contaminant neutralizing unit within the field environment, the contaminant neutralizing unit including:
a tank containing water;
an ozone generator coupled to the tank;
a nozzle coupled to the tank; and
a pump fluidly coupled to the tank and the nozzle;
supplying, by the ozone generator, ozone into the tank while the contaminant neutralizing unit is present within the field environment;
holding, by a hand of an outdoorsman, the contaminant neutralizing unit with the nozzle oriented toward the piece of outdoor equipment; and
activating the pump to move the water and the ozone from the tank, through the nozzle, and onto the piece of outdoor equipment in the field environment,
wherein the field environment is an outdoor environment containing the outdoorsman and an animal sought by the outdoorsman.

15. The method of claim 14, wherein activating the pump includes engaging, by the hand of the outdoorsman, a trigger coupled to the pump.

16. The method of claim 14, further comprising:
supplying additional water into the tank after activating the pump; and
activating the pump to move the additional water from the tank, through the nozzle, and onto a second piece of outdoor equipment in the field environment.

17. The method of claim 14, wherein the piece of outdoor equipment includes clothing configured to be worn by the outdoorsman.

18. The method of claim 14, wherein the piece of outdoor equipment includes at least one of a bow, a firearm, an arrow, or ammunition.

19. The method of claim 14, wherein the piece of outdoor equipment includes a structure configured to support or conceal the outdoorsman.

20. A method of concealing outdoor equipment in a field environment, comprising:
providing a handheld spray unit in the field environment, the handheld spray unit including a tank containing water;
applying a voltage across a pair of electrodes in contact with the water to generate ozone that mixes with the water in the tank while the handheld spray unit is present in the field environment;
stopping the generation of the ozone in response to at least one of:
an indication that a predetermined period of time has elapsed since beginning the generation of the ozone;
an indication that a concentration of ozone within the tank has reached a threshold concentration; or
an indication that a threshold level of water is no longer present in the ozone spray generating unit;
holding, by a hand of an outdoorsman, the handheld spray unit such that a nozzle of the handheld spray unit is oriented toward the outdoor equipment;
spraying, from the nozzle, the water and the ozone onto the outdoor equipment in the field environment such that the ozone reduces an odor associated with the outdoor equipment,
wherein the field environment is an outdoor environment containing an animal sought by the outdoorsman.
